(19) United States
(12) Patent Application Publication
(10) Pub. No.: US 2026/0191579 A1
(43) Pub. Date: Jul. 9, 2026

(54) ISOLATION BUMPS ON END EFFECTOR ELECTRODES

(71) Applicant: CILAG GMBH INTERNATIONAL, Zug (CH)

(72) Inventors: Jeff CLARK, Blue Ash, OH (US); Guowei John ZHANG, Cincinnati, OH (US); Steve SMOLIK, Cincinnati, OH (US)

(73) Assignee: CILAG GMBH INTERNATIONAL, Zug (CH)

(21) Appl. No.: 19/009,383

(22) Filed: Jan. 3, 2025

(51) Int. Cl. A61B 18/14; A61B 17/00; A61B 17/29; A61B 18/00; A61B 90/00

(52) U.S. Cl. CPC A61B 18/1445; A61B 2017/00526; A61B 2017/2926; A61B 2018/00428; A61B 2018/0063; A61B 2018/1455; A61B 2090/035

(57) ABSTRACT

An end effector for a surgical tool includes opposing first and second jaws, the first jaw including an electrode, and one or more isolation bumps formed on an inner surface of the electrode. Each isolation bump includes a pillar extending from the inner surface of the electrode and terminating in a plateau surface, and a curable material applied to and protruding from the plateau surface, wherein the one or more isolation bumps help define a jaw gap between the opposing first and second jaws when the opposing first and second jaws are moved to a closed position.

### BACKGROUND

Minimally invasive surgical (MIS) instruments are often preferred over traditional open surgical devices due to reduced post-operative recovery time and minimal scarring. Laparoscopic surgery is one type of MIS procedure in which one or more small incisions are formed in the abdomen of a patient and a trocar is inserted through the incision to form a pathway that provides access to the abdominal cavity. Through the trocar, a variety of instruments and surgical tools can be introduced into the abdominal cavity. The instruments and tools introduced into the abdominal cavity via the trocar can be used to engage and/or treat tissue in a number of ways to achieve a diagnostic or therapeutic effect.

Various robotic systems have recently been developed to assist in MIS procedures. Robotic systems can allow for more instinctive hand movements by maintaining natural eye-hand axis. Robotic systems can also allow for more degrees of freedom in movement by including an articulable “wrist” joint that creates a more natural hand-like articulation. In such systems, an end effector positioned at the distal end of the instrument can be articulated (moved) using a cable driven motion system having one or more drive cables (or other elongate members) that extend through the wrist joint. A user (e.g., a surgeon) is able to remotely operate the end effector by grasping and manipulating in space one or more controllers that communicate with a tool driver coupled to the surgical instrument. User inputs are processed by a computer system incorporated into the robotic surgical system, and the tool driver responds by actuating the cable driven motion system and thereby actively controlling the tension balance in the drive cables. Moving the drive cables articulates the end effector to desired angular positions and configurations.

Some end effectors have actuatable opposing jaws designed to undertake various operations during use. One type of end effector with opposing jaws, for instance, is a combination tissue grasper and vessel sealer with jaws configured to open and close to grasp onto tissue, cut through the tissue, and seal the cut tissue through electrocautery means. The gap between the opposing jaws when fully closed, referred to herein as “jaw gap,” is critical to effective operation of the tissue grasper and vessel sealer in creating proper tissue seals. If the jaw gap exceeds predetermined manufacturing tolerances by just a few thousands of an inch, the jaws may be incapable of properly sealing tissue. In such cases, the end effector will be unfit for its intended purpose and may be scrapped as a total loss.

Jaw gap is typically set during manufacture and assembly of the end effector, and must take into account manufacturing tolerances that are inherent in the individual components of the end effector. Setting the jaw gap can thus rely upon low geometric variations and tight tolerance control to produce reliable end effectors. Accordingly, methods of consistently and accurately setting jaw gap on end effectors with opposing jaws are desirable.

### BRIEF DESCRIPTION OF THE DRAWINGS

The following figures are included to illustrate certain aspects of the present disclosure, and should not be viewed as exclusive embodiments. The subject matter disclosed is capable of considerable modifications, alterations, combinations, and equivalents in form and function, without departing from the scope of this disclosure.

FIG. 1 is a block diagram of an example robotic surgical system that may incorporate some or all of the principles of the present disclosure.

FIG. 2 is an isometric side view of an example surgical tool that may incorporate some or all of the principles of the present disclosure.

FIG. 3 illustrates potential degrees of freedom in which the wrist of the surgical tool of FIG. 2 may be able to articulate (pivot) or translate.

FIG. 4 is an enlarged isometric view of the distal end of the surgical tool of FIG. 2.

FIG. 5 is an enlarged side view of the end effector of FIG. 2, according to one or more embodiments.

FIG. 6 is a schematic top view of the lower jaw with isolation bumps, according to an embodiment consistent with the present disclosure.

FIGS. 7A-7B are schematic side views of a pillar defined on an electrode during formation of an isolation bump, according to an embodiment consistent with the present disclosure.

FIGS. 8A-8D depict alternate designs of the plateau surface that produce variously-shaped pillars, according to embodiments consistent with the present disclosure

### DETAILED DESCRIPTION

The present disclosure is related to robotic surgical systems and, more particularly, methods and systems for creating a reliable and robust jaw gap between opposing jaws of a surgical tool.

Embodiments described herein disclose systems and methods of manufacturing isolation bumps and setting a jaw gap of an end effector. In the disclosed embodiments, a pillar is defined extending vertically from an inner surface of an electrode on one or more jaws of the end effector. The protruding surface may include a plateau surface at a top thereof, which may provide a flat interface on which a curable material may be applied. The curable material may be applied as a flowable slurry of material which can spread along the plateau surface and bead up at an edge defined around the plateau surface. The curable material may include a surface tension and viscosity that enables the beading of the material into a rounded, convex shape. The curable material can be cured to form isolation bumps on the inner surface of the electrode surface, which, upon pivoting the jaws of the end effector closed, may define a jaw gap therebetween. The use of the curable material on the defined plateau surface can enable finely tuned size, placement, height, and tolerances of the cured isolation bump, while also self-adhering to the electrode surface.

The isolation bumps may be positioned along a length of one or both jaws of the end effector, such that the jaw gap may be maintained or adjusted along said length. In some embodiments, more distally-formed isolation bumps may be smaller in size than the more proximally-formed isolation bumps to provide a tapered jaw gap along the length of the end effector. In further embodiments, the more distally-formed isolation bumps may be larger in size than the more proximally-formed isolation bumps, while in further embodiments still all isolation bumps may be consistently sized. The plateau surfaces and pillars can further be tuned to provide a variety of shapes and sizes of the final isolation bumps, such that oblong or complex geometries can be achieved for the plateau surfaces.

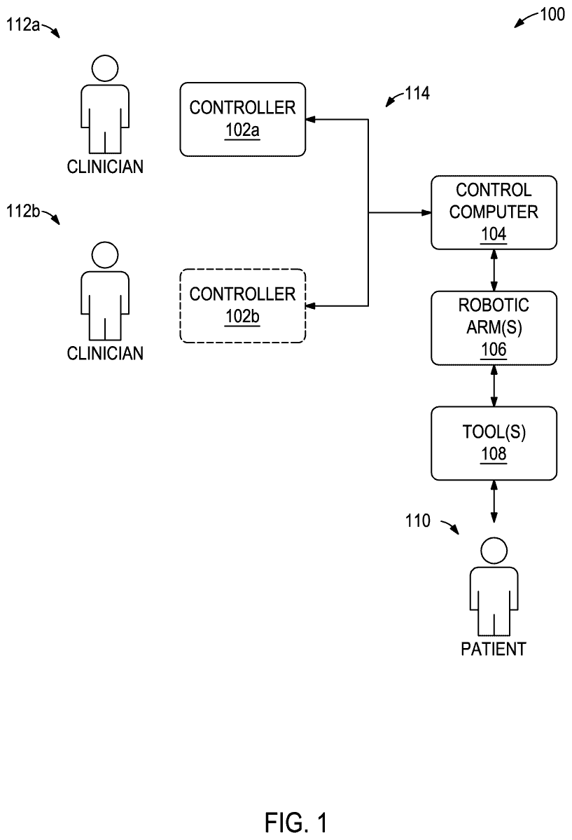

FIG. 1 is a block diagram of an example robotic surgical system 100 that may incorporate some or all of the principles of the present disclosure. As illustrated, the system 100 can include at least one set of user input controllers 102a and at least one control computer 104. The control computer 104 may be mechanically and/or electrically coupled to a robotic manipulator and, more particularly, to one or more robotic arms 106 (alternately referred to as “tool drivers”). In some embodiments, the robotic manipulator may be included in or otherwise mounted to an arm cart capable of making the system portable. Each robotic arm 106 may include and otherwise provide a location for mounting one or more surgical instruments or tools 108 for performing various surgical tasks on a patient 110. Operation of the robotic arms 106 and associated tools 108 may be directed by a clinician 112a (e.g., a surgeon) from the user input controller 102a.

In some embodiments, a second set of user input controllers 102b (shown in dashed lines) may be operated by a second clinician 112b to direct operation of the robotic arms 106 and tools 108 in conjunction with the first clinician 112a. In such embodiments, for example, each clinician 112a, b may control different robotic arms 106 or, in some cases, complete control of the robotic arms 106 may be passed between the clinicians 112a, b. In some embodiments, additional robotic manipulators (not shown) having additional robotic arms (not shown) may be utilized during surgery on the patient 110, and these additional robotic arms may be controlled by one or more of the user input controllers 102a, b.

The control computer 104 and the user input controllers 102a, b may be in communication with one another via a communications link 114, which may be any type of wired or wireless telecommunications means configured to carry a variety of communication signals (e.g., electrical, optical, infrared, etc.) and according to any communications protocol.

The user input controllers 102a, b generally include one or more physical controllers that can be grasped by the clinician 112a, b and manipulated in space while viewing the procedure via a stereo display. The physical controllers generally comprise manual input devices movable in multiple degrees of freedom, and often include an actuatable handle or pedal for actuating the surgical tool(s) 108. The control computer 104 can also include an optional feedback meter viewable by the clinician 112a, b via a display to provide a visual indication of various surgical instrument metrics, such as the amount of force being applied to the surgical instrument (i.e., a cutting instrument or dynamic clamping member).

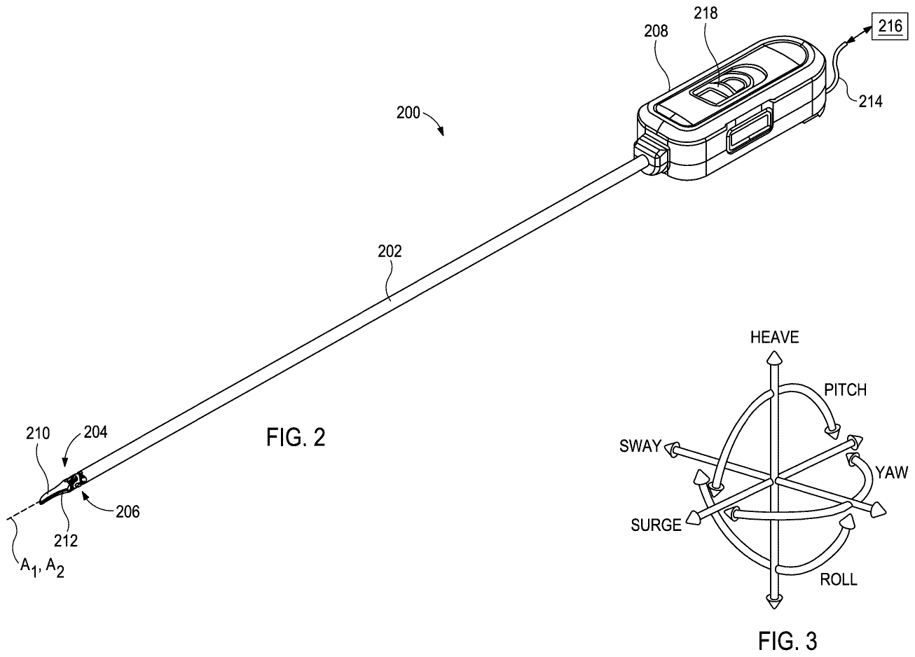

FIG. 2 is an isometric side view of an example surgical tool 200 that may incorporate some or all of the principles of the present disclosure. The surgical tool 200 may be the same as or similar to the surgical tool(s) 108 of FIG. 1 and, therefore, may be used in conjunction with a robotic surgical system, such as the robotic surgical system 100 of FIG. 1. In other embodiments, however, aspects of the surgical tool 200 may be adapted for use in a manual or hand-operated manner, without departing from the scope of the disclosure.

As illustrated, the surgical tool 200 includes an elongated shaft 202, an end effector 204, a wrist 206 (alternately referred to as a “wrist joint” or an “articulable wrist joint”) that couples the end effector 204 to the distal end of the shaft 202, and a drive housing 208 coupled to the proximal end of the shaft 202. In robotic surgical systems, the drive housing 208 can include coupling features that releasably couple the surgical tool 200 to a robotic surgical system (e.g., the robotic arm 106 of FIG. 1).

The terms “proximal” and “distal” are defined herein relative to a robotic surgical system having an interface configured to mechanically and electrically couple the surgical tool 200 (e.g., the drive housing 208) to a robotic manipulator. The term “proximal” refers to the position of an element closer to the robotic manipulator and the term “distal” refers to the position of an element closer to the end effector 204 and thus further away from the robotic manipulator. Alternatively, in manual or hand-operated applications, the terms “proximal” and “distal” are defined herein relative to a user, such as a surgeon or clinician. The term “proximal” refers to the position of an element closer to the user and the term “distal” refers to the position of an element closer to the end effector 204 and thus further away from the user. Moreover, the use of directional terms such as above, below, upper, lower, upward, downward, left, right, and the like are used in relation to the illustrative embodiments as they are depicted in the figures, the upward or upper direction being toward the top of the corresponding figure and the downward or lower direction being toward the bottom of the corresponding figure.

During use of the surgical tool 200, the end effector 204 is configured to move (pivot) relative to the shaft 202 at the wrist 206 to position the end effector 204 at desired orientations and locations relative to a surgical site. To accomplish this, the drive housing 208 includes (contains) various drive inputs and mechanisms (e.g., gears, actuators, etc.) designed to control operation of various features associated with the end effector 204 (e.g., clamping, firing, rotation, articulation, cutting, etc.). In at least some applications, the shaft 202, and hence the end effector 204 coupled thereto, is configured to rotate about a longitudinal axis A₁ of the shaft 202. In such embodiments, at least one of the drive inputs controls rotational movement of the shaft 202 about the longitudinal axis A₁.

The surgical tool 200 may include, but is not limited to, forceps, a grasper, a needle driver, scissors, an electro cautery tool, a vessel sealer, a stapler, a clip applier, a hook, a spatula, a suction tool, an irrigation tool, an imaging device (e.g., an endoscope or ultrasonic probe), or any combination thereof. In some embodiments, the surgical tool 200 may be configured to apply energy to tissue, such as radio frequency (RF) energy. In the illustrated embodiment, the end effector 204 comprises a tissue grasper and vessel sealer that includes opposing jaws 210, 212 configured to move (articulate) between open and closed positions. As will be appreciated, however, the opposing jaws 210, 212 may alternatively form part of other types of end effectors such as, but not limited to, surgical scissors, a clip applier, a needle driver, a babcock including a pair of opposed grasping jaws, bipolar jaws (e.g., bipolar Maryland grasper, forceps, a fenestrated grasper, etc.), etc. One or both of the jaws 210, 212 may be configured to pivot relative to the other to open and close the jaws 210, 212.

FIG. 3 illustrates the potential degrees of freedom in which the wrist 206 may be able to articulate (pivot). The wrist 206 comprises a joint configured to allow pivoting movement of the end effector 204 relative to the shaft 202. The degrees of freedom of the wrist 206 are represented by three translational variables (i.e., surge, heave, and sway) and three rotational variables (i.e., Euler angles or roll, pitch, and yaw). The translational and rotational variables describe the position and orientation of the end effector 204 with respect to a given reference Cartesian frame. “Surge” refers to forward and backward translational movement, “heave” refers to translational movement up and down, and “sway” refers to translational movement left and right. “Roll” refers to tilting side to side, “pitch” refers to tilting forward and backward, and “yaw” refers to turning left and right.

The pivoting motion can include pitch movement about a first axis of the wrist 206 (e.g., X-axis), yaw movement about a second axis of the wrist 206 (e.g., Y-axis), and combinations thereof to allow for 360° rotational movement of the end effector 204 about the wrist 206. In other applications, the pivoting motion can be limited to movement in a single plane, e.g., only pitch movement about the first axis of the wrist 206 or only yaw movement about the second axis of the wrist 206, such that the end effector 204 moves only in a single plane.

Referring again to FIG. 2, the surgical tool 200 may also include a plurality of drive cables (obscured in FIG. 2) that form part of a cable driven motion system that facilitates movement and articulation of the end effector 204 relative to the shaft 202. Moving (actuating) the drive cables moves the end effector 204 between an unarticulated position and an articulated position. The end effector 204 is depicted in FIG. 2 in the unarticulated position where a longitudinal axis A₂ of the end effector 204 is substantially aligned with the longitudinal axis A₁ of the shaft 202, such that the end effector 204 is at a substantially zero angle relative to the shaft 202. In the articulated position, the longitudinal axes A₁, A₂ would be angularly offset from each other such that the end effector 204 is at a non-zero angle relative to the shaft 202.

In some embodiments, the surgical tool 200 may be supplied with electrical power (current) via a power cable 214 coupled to the drive housing 208. In other embodiments, the power cable 214 may be omitted and electrical power may be supplied to the surgical tool 200 via an internal power source, such as one or more batteries or fuel cells. In such embodiments, the surgical tool 200 may alternatively be characterized and otherwise referred to as an “electrosurgical instrument” capable of providing electrical energy to the end effector 204. The power cable 214 may place the surgical tool 200 in communication with a generator 216 that supplies energy, such as electrical energy (e.g., radio frequency energy), ultrasonic energy, microwave energy, heat energy, or any combination thereof, to the surgical tool 200 and, more particularly, to the end effector 204.

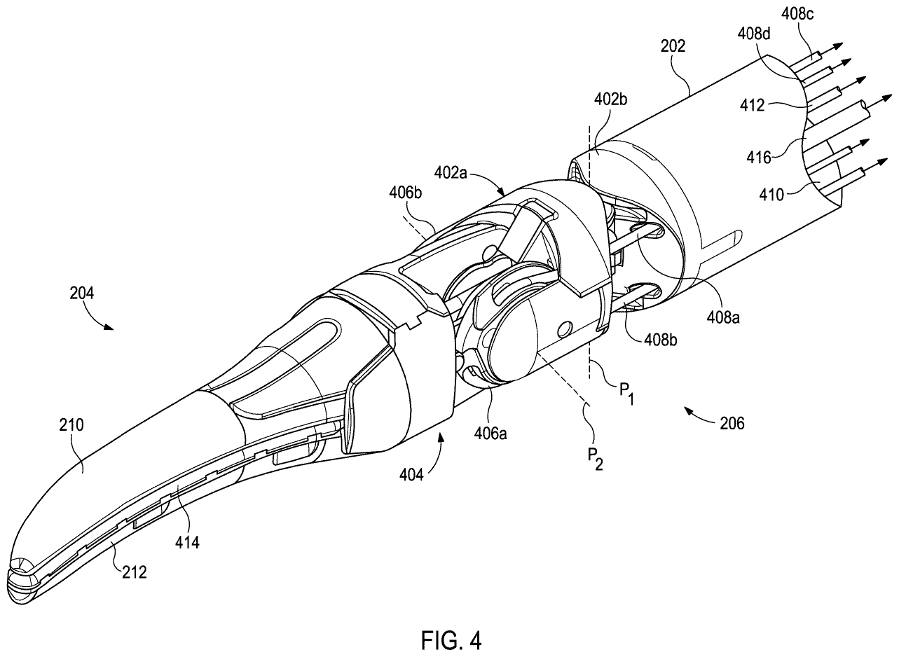

FIG. 4 is an enlarged isometric view of the distal end of the surgical tool 200 of FIG. 2. More specifically, FIG. 4 depicts an enlarged view of the end effector 204 and the wrist 206, with the jaws 210, 212 of the end effector 204 in the open position. The wrist 206 operatively couples the end effector 204 to the shaft 202. In some embodiments, however, a shaft adapter may be directly coupled to the wrist 206 and otherwise interpose the shaft 202 and the wrist 206. Accordingly, the wrist 206 may be operatively coupled to the shaft 202 either through a direct coupling engagement where the wrist 206 is directly coupled to the distal end of the shaft 202, or an indirect coupling engagement where a shaft adapter interposes the wrist 206 and the distal end of the shaft 202. As used herein, the term “operatively couple” refers to a direct or indirect coupling engagement between two components.

To operatively couple the end effector 204 to the shaft 202, the wrist 206 includes a first or “distal” clevis 402a and a second or “proximal” clevis 402b. The clevises 402a, b are alternatively referred to as “articulation joints” of the wrist 206 and extend from the shaft 202, or alternatively a shaft adapter. The clevises 402a, b are operatively coupled to facilitate articulation of the wrist 206 relative to the shaft 202. As illustrated, the wrist 206 also includes a linkage 404 arranged distal to the distal clevis 402a and operatively mounted to the jaws 210, 212.

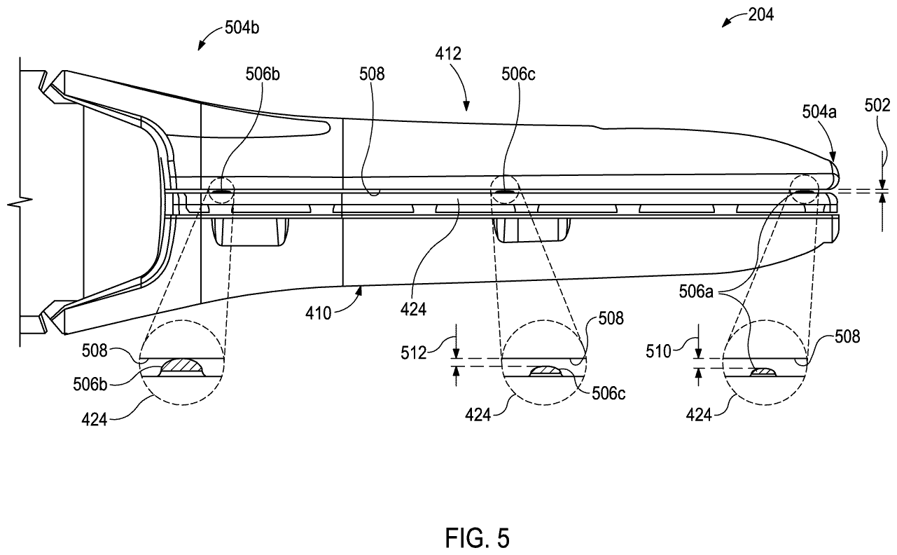

As illustrated, the proximal end of the distal clevis 402a may be rotatably mounted or pivotably coupled to the proximal clevis 402b at a first pivot axis P₁ of the wrist 206. In some embodiments, an axle may extend through the first pivot axis P₁ and the distal and proximal clevises 402a, b may be rotatably coupled via the axle. In other embodiments, however, such as is depicted in FIG. 5, the distal and proximal clevises 402a, b may be engaged in rolling contact, such as via an intermeshed gear relationship that allows the clevises 402a, b to rotate relative to each other similar to a rolling joint.

First and second pulleys 406a and 406b may be rotatably mounted to the distal end of the distal clevis 402a at a second pivot axis P₂ of the wrist 206. The linkage 404 may be arranged distal to the second pivot axis P₂ and operatively mounted to the jaws 210, 212. The first pivot axis P₁ is substantially perpendicular (orthogonal) to the longitudinal axis A₁ of the shaft 202, and the second pivot axis P₂ is substantially perpendicular (orthogonal) to both the longitudinal axis A₁ and the first pivot axis P₁. Movement of the end effector 204 about the first pivot axis P₁ provides “yaw” articulation of the wrist 206, and movement about the second pivot axis P₂ provides “pitch” articulation of the wrist 206.

A plurality of drive cables, shown as drive cables 408a, 408b, 408c, and 408d, extend longitudinally within a lumen 410 defined by the shaft 202 (or a shaft adaptor) and pass through the wrist 206 to be operatively coupled to the end effector 204. The drive cables 408a-d form part of the cable driven motion system briefly described above, and may be referred to and otherwise characterized as cables, bands, lines, cords, wires, woven wires, ropes, strings, twisted strings, elongate members, etc. The drive cables 408a-d can be made from a variety of materials including, but not limited to, metal (e.g., tungsten, stainless steel, etc.) a polymer (e.g., ultra-high molecular weight polyethylene), a synthetic fiber (e.g., KEVLAR®, VECTRAN®, etc.), or any combination thereof. While four drive cables 408a-d are depicted in FIG. 4, more or less than four drive cables 408a-d may be included, without departing from the scope of the disclosure.

The drive cables 408a-d extend proximally from the end effector 204 to the drive housing 208 (FIG. 2) where they are operatively coupled to various actuation mechanisms (e.g., capstans) or devices housed therein to facilitate longitudinal movement (translation) of the drive cables 408a-d within the lumen 410. Selective actuation of the drive cables 408a-d causes corresponding drive cables 408a-d to translate longitudinally within the lumen 410. Moving a given drive cable 408a-d applies tension (i.e., pull force) to the given drive cable 408a-d in a proximal direction, which causes the given drive cable 408a-d to translate and thereby cause the end effector 204 to move (articulate).

The drive cables 408a-d each extend longitudinally through the proximal clevis 402b. The distal end of each drive cable 408a-d terminates at the first or second pulleys 406a, b, thus operatively coupling each drive cable 408a-d to the end effector 204. In some embodiments, the distal ends of the first and second drive cables 408a, b may be coupled to each other and terminate at the first pulley 406a, and the distal ends of the third and fourth drive cables 408c, d may be coupled to each other and terminate at the second pulley 406b. In at least one embodiment, the distal ends of the first and second drive cables 408a, b and the distal ends of the third and fourth drive cables 408c, d may each be coupled together at corresponding ball crimps (not shown) mounted to the first and second pulleys 406a, b, respectively.

In at least one embodiment, the drive cables 408a-d may operate “antagonistically”. More specifically, when the first drive cable 408a is actuated (moved), the second drive cable 408b naturally follows as coupled to the first drive cable 408a, and when the third drive cable 408c is actuated, the fourth drive cable 408d naturally follows as coupled to the third drive cable 408c, and vice versa. Antagonistic operation of the drive cables 408a-d can open or close the jaws 210, 212 and can further cause the end effector 204 to articulate at the wrist 206. More specifically, selective actuation of the drive cables 408a-d in known configurations or coordination can cause the end effector 204 to articulate about one or both of the pivot axes P₁, P₂, thus facilitating articulation of the end effector 204 in both pitch and yaw directions. Moreover, selective actuation of the drive cables 408a-d in other known configurations or coordination will cause the jaws 210, 212 to open or close. Antagonistic operation of the drive cables 408a-d advantageously reduces the number of cables required to provide full wrist 206 motion, and also helps eliminate slack in the drive cables 408a-d, which results in more precise motion of the end effector 204.

In the illustrated embodiment, the end effector 204 is able to articulate (move) in pitch about the second or “pitch” pivot axis P₂, which is located near the distal end of the wrist 206. Thus, the jaws 210, 212 open and close in the direction of pitch. In other embodiments, however, the wrist 206 may alternatively be configured such that the second pivot axis P₂ facilitates yaw articulation of the jaws 210, 212, without departing from the scope of the disclosure.

In some embodiments, an electrical conductor 412 may also extend longitudinally within the lumen 410, through the wrist 206, and terminate at an electrode 414 to supply electrical energy to the end effector 204. In some embodiments, the electrical conductor 412 may comprise a wire, but may alternatively comprise a rigid or semi-rigid shaft, rod, or strip (ribbon) made of a conductive material. The electrical conductor 412 may be entirely or partially covered with an insulative covering (overmold) made of a non-conductive material. Using the electrical conductor 412 and the electrode 414, the end effector 204 may be configured for monopolar or bipolar RF operation.

In the illustrated embodiment, the end effector 204 comprises a combination tissue grasper and vessel sealer that includes a knife (not visible), alternately referred to as a “cutting element” or “blade.” The knife is aligned with and configured to traverse a guide track or “knife slot” (not visible) defined longitudinally in one or both of the upper and lower jaws 210, 212. The knife may be operatively coupled to the distal end of a knife rod 416 (alternately referred to as “drive rod,” “actuation rod,” or “push rod”) that extends longitudinally within the lumen 410 and passes through the wrist 206. Longitudinal movement (translation) of the knife rod 416 correspondingly moves the knife within the knife slot(s). Similar to the drive cables 408a-d, the knife rod 416 may form part of the actuation systems housed within the drive housing 208 (FIG. 2). Selective actuation of a corresponding drive input will cause the knife rod 416 to move distally or proximally within the lumen 410, and correspondingly move the knife in the same longitudinal direction.

The knife rod 416 may comprise a rigid or semi rigid elongate member, such as a rod or shaft (e.g., a hypotube, a hollow rod, a solid rod, etc.), a wire, a ribbon, a push cable, or any combination thereof. The knife rod 416 can be made from a variety of materials including, but not limited to, metal (e.g., tungsten, nitinol, stainless steel, etc.), a polymer, or a composite material. The knife rod 416 may have a circular cross-section, but may alternatively exhibit a polygonal cross-section without departing from the scope of the disclosure.

FIG. 5 is an enlarged side view of the end effector 204 of FIGS. 2 and 4, according to one or more embodiments. The jaws 210, 212 are shown in the closed position and are slightly offset from each other such that a jaw gap 502 is defined between the inner (opposing) surfaces of each jaw 210, 212. As mentioned above, the jaw gap 502 is critical to effective operation of the end effector 204 in creating proper tissue seals. For instance, the magnitude of the jaw gap 502 can be tied to a predetermined manufacturing specification value, and if the jaw gap 502 exceeds the predetermined value by just a few thousands of an inch (in either direction), the jaws 210, 212 may be incapable of properly sealing, and cutting tissue, and thus may be unfit for its intended purpose.

In some embodiments, the jaw gap 502 may be generally uniform along the proximal-to-distal (longitudinal) length of the jaws 210, 212 such that the inner surfaces of each jaw 210, 212 are substantially parallel to one another when closed. In other embodiments, however, the inner surfaces of each jaw 210, 212 are may be non-parallel and the jaw gap 502 may thus be non-uniform along the longitudinal length to enhance sealing performance. In the illustrated embodiment, for example, the magnitude of the jaw gap 502 increases in the proximal direction, from a distal end 504a of the jaws 210, 212 toward a proximal end 504b of the jaws 210, 212.

According to embodiments of the present disclosure, the end effector 204 includes a plurality of isolation bumps 506 that ensures the inner surfaces of the jaws 210, 212 do not touch during operation and when the jaws 210, 212 move to the closed position. More specifically, the end effector 204 may include one or more distal isolation bumps 506a provided at or near the distal end 504a, one or more proximal isolation bumps 506b provided at or near the proximal end 504b, and one or more intermediate isolation bumps 504c provided at a location between the distal and proximal ends 504a, b.

In some embodiments, the isolation bumps 506a-c may be formed on an upper surface of the electrode 424 of the lower jaw 210. In other embodiments, however, the isolation bumps 506a-c may extend from an inner surface 508 of the upper jaw 212 and toward the electrode 424, without departing from the scope of the disclosure. As shown in FIG. 5, the isolation bumps 506a-c may extend from the surface of the electrode 424 provided on the lower jaw 210 to engage the inner surface 508 of the upper jaw 212. In yet other embodiments, the isolation bumps 506a-c may extend from a combination of the electrode 424 and the inner surface 508, without departing from the scope of the disclosure.

During assembly of the end effector 204, the jaw gap 502 may be set by first moving the jaws 210, 212 to the closed position until the proximal isolation bump(s) 506b engage the inner surface 508 at the proximal end 504b, as shown in the enlarged inset graphic. The jaws 210, 212 may then be progressively closed toward the distal end 504a. In at least one embodiment, however, the jaw gap 502 may be set such that the magnitude of the jaw gap 502 at the distal end 504a is greater than the magnitude of the jaw gap 502 at the proximal end 504b. In such embodiments, a non-zero angle will be formed between the inner surface 508 of the upper jaw 212 and the electrode 424. The angle may be sufficient such that a distal gap 510 is formed between the distal isolation bump(s) 506b and the inner surface 508, as shown in the enlarged inset graphic.

In some embodiments, an intermediate gap 512 may also be formed between the intermediate isolation bump(s) 506c and the inner surface 508, as shown in the enlarged inset graphic. In alternate embodiments, however, the distal isolation bump(s) 506a may contact the inner surface 508 to maintain tip-first closure of the jaws 210, 212, while the proximal isolation bump(s) 506b present a proximal gap (not shown) relative to the inner surface 508, without departing from the scope of this disclosure. Accordingly, the isolation bumps 506a-c enable different heights, which can be selectively adjusted to achieve a desired jaw gap 502.

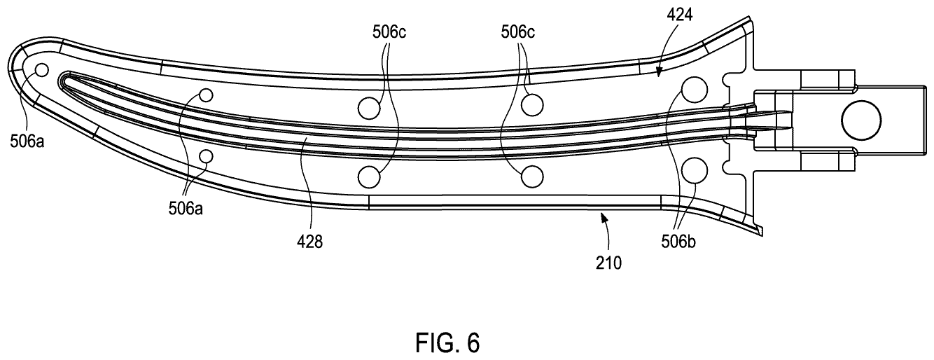

FIG. 6 is a schematic top view of a portion of the lower jaw 210 with the isolation bumps 506a-c defined thereon, according to an embodiment consistent with the present disclosure. As illustrated, a plurality of isolation bumps 506a-c can be provided (defined) along a longitudinal length of the electrode 424 such that the jaw gap 502 (FIG. 5) may be maintained or adjusted along the length of the lower jaw 210. In some embodiments one or more of the isolation bumps 506a-c may be provided in pairs along the longitudinal length of the lower jaw 210. In such embodiments, an isolation bump 506a-c may be defined on each side of the guide track 428 and may be generally aligned along the longitudinal length of the electrode 424. The pairs of isolation bumps 506a-c may maintain the jaw gap across the width of the end effector 204 (FIGS. 2, 4-5) to prevent axial rotation or slippage about the guide track 428 during use.

As discussed above, the lower jaw 210 may include one or more proximal isolation bumps 506b, which may be larger (e.g., in shape, volume, height, etc.) than the one or more distal isolation bumps 506a. In the illustrated embodiment, the one or more intermediate isolation bumps 506c may exhibit a size (volume) between that of the proximal and distal isolation bumps 506a, b to provide a consistent transition and angled jaw gap 502 (FIG. 5) along the length of the lower jaw 210. In alternate embodiments, however, the one or more distal isolation bumps 506a may be of a larger (e.g., in shape, volume, height, etc.) than that of the one or more proximal isolation bumps 506b. In yet other embodiments, each of the isolation bumps 506a-c may exhibit the same size to maintain the same jaw gap 502 across the length of the end effector 204 (FIGS. 2, 4, and 5), without departing from the scope of the present disclosure.

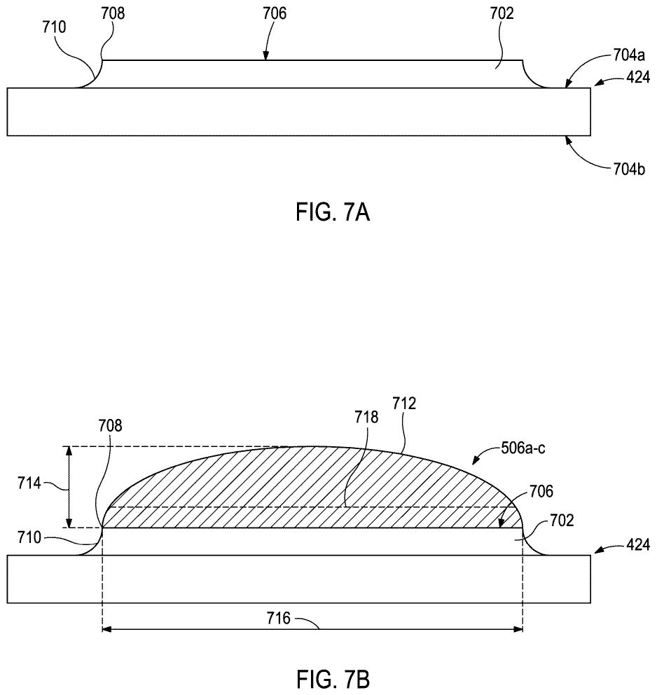

FIGS. 7A and 7B are side views of a portion of the electrode 424 depicting example formation (creation) of an example isolation bump 506, according to one or more embodiments of the present disclosure. The isolation bump 506 may represent any of the isolation bumps 506a-c shown in FIG. 5 or 6.

Referring first to FIG. 7A, a plateau feature or “pillar” 702 can be formed on the electrode 424 and protrude (extend) vertically from an inner surface 704a of the electrode 424. The pillar 702 may be formed to define a plateau surface 706 vertically offset from the inner surface 704a of the electrode 424. In some embodiments, the plateau surface 706 may be flat (smooth) and parallel to the inner surface 704a of the electrode 424. In other embodiments, however, the plateau surface 706 may be non-flat (e.g., undulating, rough, etc.) and/or non-parallel to the inner surface 704a.

In some embodiments, the pillar 702 can be defined on the electrode 424 via photochemical machining process, which removes portions of the inner surface 704a of the electrode 424 to thereby form the geometry of the pillar 702. Removing adjacent material via photochemical machining can enable precise etching of the pillar 702 with fine control of size, depth, and positioning of the pillar 702 on the inner surface 704a of the electrode 424. In other embodiments, however, the pillar 702 may be formed using a die tool or punch (not shown). In such embodiments the die tool may be used to punch the shape of the pillar 702 from an outer (underside) surface 704b of the electrode 424 such that the pillar 702 extends away from and is otherwise formed on the inner surface 704a. In such embodiments, the die tool may be provided in a desired size and shape to dimple the electrode 424 in the size and shape of the pillar 702, while maintaining an overall thickness of the electrode 424. Other methods of manufacturing contemplated herein include, but are not limited to, electrochemical machining (ECM), computer numerical control (CNC) machining, metal injection molding (MIM), laser machining, or a combination of any of the foregoing. These methods of manufacturing the pillar 702 may provide tight tolerances and fine control of the shape and size of the resulting plateau surface 706, as well as consistent production of pillars along the electrode 424.

The plateau surface 706 may be defined such that an edge 708 is defined around (circumscribes) the plateau surface 706. The edge 708 may form the perimeter and a distinct lip about the transition surface 710, such that a liquid or slurry can be retained thereon via surface tension, as discussed below. The transition surface 710 extends between the edge 708 and the inner surface 704a of the electrode 424. In some embodiments, as illustrated, the transition surface 710 may be curved, arcuate, and otherwise define a sloping chamfer. This may prove advantageous in helping to prevent sharp edges or corners on the electrode 424 that could snag or shear tissue during operation of the end effector 204 (FIGS. 2, 4, and 5). In other embodiments, however, the transition surface 710 may be straight (vertically) and otherwise substantially orthogonal to the inner surface 704a of the electrode 424, without departing from the scope of the disclosure.

In FIG. 7B a curable material 712 is applied to the top of the plateau surface 706 and allowed to cure. In some embodiments, the curable material 712 may be applied to the plateau surface 706 as a slurry and may comprise a material that possesses high dielectric strength, high compressive strength, and high yield stress. The curable material 712 can include a slurry that includes, for example, a ceramic paste, a glass paste, a polymer, or any combination thereof.

As a slurry, the curable material 712 can be deposited on the plateau surface 706 and allowed to flow towards the edge 708 circumscribing the plateau surface 706. The slurry of the curable material 712 may exhibit a surface tension of sufficient force that allows the curable material 712 to form bead or dome-like geometry on the plateau surface 706 that extends to defined edge 708. The surface tension and viscosity of the curable material 712 may accordingly control a height 714 of the bead and thereby form a convex shape. The height 714 of the resulting isolation bump 506a-c may also depend on a width 716 of the plateau surface 706, such that the surface tension of the slurry of curable material 712 can define an aspect ratio between the height 714 and width 716 of the isolation bump 506a-c. As such, the deposition of the curable material 712 may be performed via an automated system to provide a precise amount of curable material 712 to form the desired shape of the isolation bump 506a-c without overflowing past the edge 708 of the plateau surface 706.

In some embodiments, the curable material 712 may be cured (e.g., in an oven) at a temperature ranging from about 800° C. to about 1000° C. During curing, the slurry of the curable material 712 may mechanically interlock with the plateau surface 706 at the grain structure therebetween. In further embodiments, the plateau surface 706 may be treated, scored, or otherwise prepared to enable mechanical interlocking between the plateau surface 706 and curable material 712, and thereby provide enhanced adhesion between the materials. In alternate embodiments, an intermediate material (not shown) may be included on the plateau surface 706 prior to deposition of the curable material 712, such that the intermediate material facilitates adhesion between the plateau surface 706 and the curable material 712.

Following curing of the curable material 712, the sharp perimeter of the edge 708 may be obfuscated by the rounded edge of the curable material 712, thereby preventing any snagging or shearing of tissue during operation of the end effector 204 (FIGS. 2, 4-5). As shown in the illustrated embodiment, a smooth, rounded interface may be accordingly defined between the transition surface 710 and the curable material 712.

In some embodiments, as illustrated, the curable material 712 forms a rounded, convex shape and upper surface when it fully cures. The rounded, convex shape may result from the surface tension of the curable material 712, which causes the curable material 712 to form a bead or dome-like geometry. In other embodiments, however, the curable material 712 may form a flat upper surface 718 when it fully cures. In at least one embodiment, the flat upper surface 718 may result from machining the curable material 712 to form a planar, smooth surface.

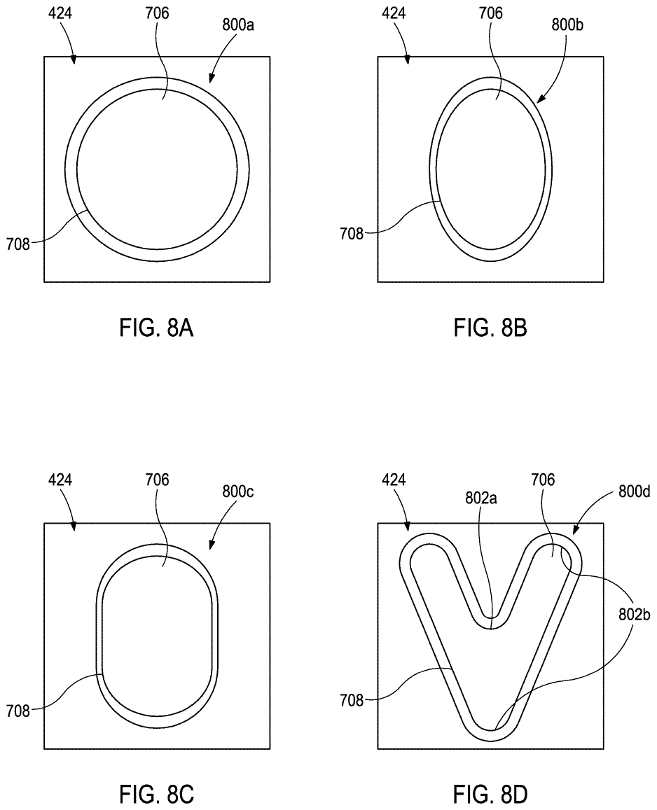

FIGS. 8A-8D are schematic top views of example plateau surfaces 706 that produce variously-shaped pillars 800a-d, according to embodiments consistent with the present disclosure. Referring first to FIG. 8A, the pillar 800a may provide an approximately circular cross-section or geometry. The pillar 800a may be shaped such that the edge 708 forms a consistent, curved boundary without any sharp edges or sharp corners. In such embodiments, the curable material 712 (FIG. 7B) may form a partially-spherical shape or dome on the plateau surface 706.

In FIGS. 8B and 8C, the pillars 800b and 800c may provide rounded, but non-circular shapes, such that an elliptical feature and an obround feature may be defined, respectively. The pillars 800b-c may provide elongated shapes to alter the aspect ratio of the plateau surface 706, while the edge 708 includes rounded corners and no sharp angles to maintain surface tension on the plateau surface 706. Another non-circular shape contemplated herein is ovoid.

In FIG. 8D, the pillar 800d may be generally polygonal in shape, but no sharp corners are provided. More specifically, the pillar 800d generally defines a V-shaped plateau surface 706, such that the resulting isolation bump 506a-c (FIG. 5) may be irregularly formed on the electrode 424. The interior corners 802a and the exterior corners 802b of the V-shaped plateau surface 706 may be smoothly rounded to maintain the edge 708 without sharp corners or angles. As such, the plateau surface 706 can be formed in any irregular shape, provided that both the interior corners 802a and exterior corners 802b of any defined shape are accordingly rounded. Other polygonal shapes without sharp corners or angles are also contemplated herein, such as triangular, rectangular, pentagonal, etc., without departing from the scope of the disclosure.

Embodiments disclosed herein include:
A. An end effector for a surgical tool including opposing first and second jaws, the first jaw including an electrode, and one or more isolation bumps formed on an inner surface of the electrode. Each isolation bump includes a pillar extending from the inner surface of the electrode and terminating in a plateau surface, and a curable material applied to and protruding from the plateau surface, wherein the one or more isolation bumps help define a jaw gap between the opposing first and second jaws when the opposing first and second jaws are moved to a closed position.
B. A method of manufacturing an isolation bump on an electrode of an end effector, the method including forming a pillar on an inner surface of an electrode of a first jaw of the end effector, the end effector further including a second jaw opposing the first jaw, and the pillar defining a plateau surface circumscribed by an edge, applying a slurry of a curable material to the plateau surface, and curing the slurry on the plateau surface and thereby forming an isolation bump.
C. A method of setting a jaw gap of an end effector including forming a pillar on an inner surface of an electrode of a first jaw of the end effector, the end effector further including a second jaw opposing the first jaw, and the pillar defining a plateau surface circumscribed by an edge, applying a slurry of a curable material to the plateau surface, curing the slurry on the plateau surface and thereby forming an isolation bump, and moving the first and second jaws toward a closed position and thereby engaging an inner surface of the second jaw with the isolation bump to define the jaw gap between the first and second jaws.

Each of embodiments A through C may have one or more of the following additional elements in any combination: Element 1: wherein each isolation bump further includes a transition surface extending between an edge of the plateau surface and the inner surface of the electrode, and wherein the transition surface is curved or arcuate. Element 2: wherein the curable material is selected from the group consisting of a ceramic, a glass, a polymer, and any combination thereof. Element 3: wherein the one or more isolation bumps comprise a plurality of isolation bumps defined along a length of the first jaw and including one or more distally-located isolation bumps provided at or near a distal end of the first jaw; and one or more proximally-located isolation bumps provided at or near a proximal end of the first jaw, wherein the one or more distally-located isolation bumps exhibit a height less than the one or more proximally-located isolation bumps. Element 4: wherein the one or more isolation bumps comprise a plurality of isolation bumps defined along a length of the first jaw and including one or more distally-located isolation bumps provided at or near a distal end of the first jaw; and one or more proximally-located isolation bumps provided at or near a proximal end of the first jaw, wherein the one or more distally-located isolation bumps exhibit a height greater than the one or more proximally-located isolation bumps. Element 5: wherein the plateau surface exhibits a shape without sharp corners or angles. Element 6:, wherein the shape of the plateau surface is selected from the group consisting of circular, oval, obround, ovoid, polygonal, and any combination thereof. Element 7: wherein forming the pillar comprises using photochemical machining to remove portions of the electrode around the pillar. Element 8: wherein forming the pillar comprises punching a dimple from an outer surface of the electrode.

Element 9: wherein the curable material is selected from the group consisting of a ceramic, a glass, a polymer, and any combination thereof. Element 10: wherein curing the slurry of the curable material comprises heating the slurry to a temperature of about 800° C. to about 1000° C. Element 11: wherein forming the pillar includes defining the plateau surface to exhibit a shape without sharp corners or angles. Element 12: wherein the plateau surface is defined to exhibit a shape selected from the group consisting of circular, oval, obround, ovoid, polygonal, and any combination thereof. Element 13: further comprising forming one or more further isolation bumps along a length of the first jaw, the one or more further isolation bumps including one or more distally-located isolation bumps provided at or near a distal end of the first jaw; and one or more proximally-located isolation bumps provided at or near a proximal end of the first jaw, wherein the one or more distally-located isolation bumps exhibit a height less than the one or more proximally-located isolation bumps. Element 14: further comprising forming one or more further isolation bumps along a length of the first jaw, the one or more further isolation bumps including one or more distally-located isolation bumps provided at or near a distal end of the first jaw; and one or more proximally-located isolation bumps provided at or near a proximal end of the first jaw, wherein the one or more distally-located isolation bumps exhibit a height greater than the one or more proximally-located isolation bumps. Element 15: wherein the slurry is retained on the plateau surface via surface tension of the slurry at an edge of the plateau surface. Element 16: wherein forming the pillar includes defining the plateau surface to exhibit a shape without sharp corners or angles. Element 17: wherein the plateau surface is defined to exhibit a shape selected from the group consisting of circular, oval, obround, ovoid, polygonal, and any combination thereof.

By way of non-limiting example, exemplary combinations applicable to A through C include: Element 1 with Element 2; Element 5 with Element 6; Element 8 with Element 9; Element 11 with Element 12; and Element 16 with Element 17.

Therefore, the disclosed systems and methods are well adapted to attain the ends and advantages mentioned as well as those that are inherent therein. The particular embodiments disclosed above are illustrative only, as the teachings of the present disclosure may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative embodiments disclosed above may be altered, combined, or modified and all such variations are considered within the scope of the present disclosure. The systems and methods illustratively disclosed herein may suitably be practiced in the absence of any element that is not specifically disclosed herein and/or any optional element disclosed herein. While compositions and methods are described in terms of “comprising,” “containing,” or “including” various components or steps, the compositions and methods can also “consist essentially of” or “consist of” the various components and steps. All numbers and ranges disclosed above may vary by some amount. Whenever a numerical range with a lower limit and an upper limit is disclosed, any number and any included range falling within the range is specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an,” as used in the claims, are defined herein to mean one or more than one of the elements that it introduces. If there is any conflict in the usages of a word or term in this specification and one or more patent or other documents that may be incorporated herein by reference, the definitions that are consistent with this specification should be adopted.

As used herein, the phrase “at least one of” preceding a series of items, with the terms “and” or “or” to separate any of the items, modifies the list as a whole, rather than each member of the list (i.e., each item). The phrase “at least one of” allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

What is claimed is:

1. An end effector for a surgical tool, comprising:
opposing first and second jaws, the first jaw including an electrode; and
one or more isolation bumps formed on an inner surface of the electrode, each isolation bump comprising:
a pillar extending from the inner surface of the electrode and terminating in a plateau surface; and
a curable material applied to and protruding from the plateau surface,
wherein the one or more isolation bumps help define a jaw gap between the opposing first and second jaws when the opposing first and second jaws are moved to a closed position.

2. The end effector of claim 1, wherein each isolation bump further includes a transition surface extending between an edge of the plateau surface and the inner surface of the electrode, and wherein the transition surface is curved or arcuate.

3. The end effector of claim 2, wherein the curable material is selected from the group consisting of a ceramic, a glass, a polymer, and any combination thereof.

4. The end effector of claim 1, wherein the one or more isolation bumps comprise a plurality of isolation bumps defined along a length of the first jaw and including:
one or more distally-located isolation bumps provided at or near a distal end of the first jaw; and
one or more proximally-located isolation bumps provided at or near a proximal end of the first jaw,
wherein the one or more distally-located isolation bumps exhibit a height less than the one or more proximally-located isolation bumps.

5. The end effector of claim 1, wherein the one or more isolation bumps comprise a plurality of isolation bumps defined along a length of the first jaw and including:
one or more distally-located isolation bumps provided at or near a distal end of the first jaw; and
one or more proximally-located isolation bumps provided at or near a proximal end of the first jaw,
wherein the one or more distally-located isolation bumps exhibit a height greater than the one or more proximally-located isolation bumps.

6. The end effector of claim 1, wherein the plateau surface exhibits a shape without sharp corners or angles.

7. The end effector of claim 6, wherein the shape of the plateau surface is selected from the group consisting of circular, oval, obround, ovoid, polygonal, and any combination thereof.

8. A method of manufacturing an isolation bump on an electrode of an end effector, the method comprising:
forming a pillar on an inner surface of an electrode of a first jaw of the end effector, the end effector further including a second jaw opposing the first jaw, and the pillar defining a plateau surface circumscribed by an edge;
applying a slurry of a curable material to the plateau surface; and
curing the slurry on the plateau surface and thereby forming an isolation bump.

9. The method of claim 8, wherein forming the pillar comprises using photochemical machining to remove portions of the electrode around the pillar.

10. The method of claim 8, wherein forming the pillar comprises punching a dimple from an outer surface of the electrode.

11. The method of claim 10, wherein the curable material is selected from the group consisting of a ceramic, a glass, a polymer, and any combination thereof.

12. The method of claim 8, wherein curing the slurry of the curable material comprises heating the slurry to a temperature of about 800° C. to about 1000°C.

13. The method of claim 8, wherein forming the pillar includes defining the plateau surface to exhibit a shape without sharp corners or angles.

14. The method of claim 13, wherein the plateau surface is defined to exhibit a shape selected from the group consisting of circular, oval, obround, ovoid, polygonal, and any combination thereof.

15. A method of setting a jaw gap of an end effector, comprising:
forming a pillar on an inner surface of an electrode of a first jaw of the end effector, the end effector further including a second jaw opposing the first jaw, and the pillar defining a plateau surface circumscribed by an edge;
applying a slurry of a curable material to the plateau surface;
curing the slurry on the plateau surface and thereby forming an isolation bump; and
moving the first and second jaws toward a closed position and thereby engaging an inner surface of the second jaw with the isolation bump to define the jaw gap between the first and second jaws.

16. The method of claim 15, further comprising forming one or more further isolation bumps along a length of the first jaw, the one or more further isolation bumps including:
one or more distally-located isolation bumps provided at or near a distal end of the first jaw; and
one or more proximally-located isolation bumps provided at or near a proximal end of the first jaw,
wherein the one or more distally-located isolation bumps exhibit a height less than the one or more proximally-located isolation bumps.

17. The method of claim 15, further comprising forming one or more further isolation bumps along a length of the first jaw, the one or more further isolation bumps including:
one or more distally-located isolation bumps provided at or near a distal end of the first jaw; and
one or more proximally-located isolation bumps provided at or near a proximal end of the first jaw,
wherein the one or more distally-located isolation bumps exhibit a height greater than the one or more proximally-located isolation bumps.

18. The method of claim 15, wherein the slurry is retained on the plateau surface via surface tension of the slurry at an edge of the plateau surface.

19. The method of claim 15, wherein forming the pillar includes defining the plateau surface to exhibit a shape without sharp corners or angles.

20. The method of claim 19, wherein the plateau surface is defined to exhibit a shape selected from the group consisting of circular, oval, obround, ovoid, polygonal, and any combination thereof.
